SEQUENCE LISTING (67 sequences)

SEQ ID NO: 1 - DNA, 23 bp, Artificial
  HIV-1 Primer
ccaggaatatggcaattagattg

SEQ ID NO: 2 - DNA, 20 bp, Artificial
  HIV-1 Primer
cctgccatctgttttccata

SEQ ID NO: 3 - DNA, 20 bp, Artificial
  HIV-1 Primer
gcagtccatgtagccagtgg

SEQ ID NO: 4 - DNA, 29 bp, Artificial
  HIV-1 Primer
cacaattttaaaagaaaaggggggattgg

SEQ ID NO: 5 - DNA, 28 bp, Artificial
  HIV-1 Primer
tagacataatagcaacagacatacaaac

SEQ ID NO: 6 - DNA, 20 bp, Artificial
  HIV-1 Primer
tattacagggacagcagaga

SEQ ID NO: 7 - DNA, 29 bp, Artificial
  HIV-1 Primer
gacagcagagacccaatttggaaaggacc

SEQ ID NO: 8 - DNA, 23 bp, Artificial
  HIV-1 Primer
tggaaaggtgaaggggcagtagt

SEQ ID NO: 9 - DNA, 23 bp, Artificial
  HIV-1 Primer
aattggagagcaatggctagtga

SEQ ID NO: 10 - DNA, 20 bp, Artificial
  HIV-1 Primer
ccttctaaatgtgtacaatc

SEQ ID NO: 11 - DNA, 25 bp, Artificial
  HIV-1 Primer
tctgctgggataacttctgcttcta

SEQ ID NO: 12 - DNA, 32 bp, Artificial
  HIV-1 Primer
ttattcatagattctactactccttgactttg

SEQ ID NO: 13 - DNA, 18 bp, Artificial
  HIV-1 Primer
aaggcagcctgttggtgg

SEQ ID NO: 14 - DNA, 28 bp, Artificial
  HIV-1 Primer
gtttgtatgtctgttgctattatgtcta

SEQ ID NO: 15 - DNA, 23 bp, Artificial
  HIV-1 Primer
actactgccccttcacctttcca

SEQ ID NO: 16 - DNA, 20 bp, Artificial
  HIV-1 Primer
gattgtacacatttagaagg

SEQ ID NO: 17 - DNA, 23 bp, Artificial
  HIV-1 Primer
aatactgccatttgtactgctgt

SEQ ID NO: 18 - DNA, 32 bp, Artificial
  HIV-1 Primer
cccccaatccccccttttcttttaaaattgtg

SEQ ID NO: 19 - DNA, 33 bp, Artificial
  HIV-1 Primer
aagatggccagtaaaagtaatacacacagacaa

SEQ ID NO: 20 - DNA, 21 bp, Artificial
  HIV-1 Primer
acccgaaaattttgaattttt

SEQ ID NO: 21 - DNA, 32 bp, Artificial
  HIV-1 Primer
caaagtcaaggagtagtagaatctatgaataa

SEQ ID NO: 22 - DNA, 20 bp, Artificial
  HIV-1 Primer
tctctgctgtccctgtaata

SEQ ID NO: 23 - DNA, 29 bp, Artificial
  HIV-1 Primer
ggtcctttccaaattgggtctctgctgtc

SEQ ID NO: 24 - DNA, 23 bp, Artificial
  HIV-1 Primer polI8
tagtgggatgtgtacttctgaac

SEQ ID NO: 25 - DNA, 24 bp, Artificial
  HIV-1 Primer polI5
cacacaaaggrattggaggaaatg

SEQ ID NO: 26 - DNA, 22 bp, Artificial
  HIV-1 Primer O-polI8
gattyctggattcataatgatg

SEQ ID NO: 27 - DNA, 21 bp, Artificial
  HIV-1 Primer O-polI5
gtatcttacatgggttcctgc

SEQ ID NO: 28 - DNA, 29 bp, Artificial
  HIV-1 Primer
attccctacaatccccaaagtcaaggagt

SEQ ID NO: 29 - DNA, 25 bp, Artificial
  HIV-1 Primer
cccctgcactgtaccccccaatccc

SEQ ID NO: 30 - DNA, 22 bp, Artificial
  HIV-1 Primer
cctgcactgtaccccccaatcc

SEQ ID NO: 31 - DNA, 29 bp, Artificial
  HIV-1 Primer
ccaatccccccttttcttttaaaattgtc

SEQ ID NO: 32 - DNA, 29 bp, Artificial
  HIV-1 Primer
tgtattactactgccccttcacctttcca

SEQ ID NO: 33 - DNA, 27 bp, Artificial
  HIV-1 Primer
atcatcacctgccatctgttttccata

SEQ ID NO: 34 - DNA, 28 bp, Artificial
  HIV-1 Primer
cacaattttaaaagaaaaggggggattg

SEQ ID NO: 35 - DNA, 28 bp, Artificial
  HIV-1 Primer
tgtattactactgccccttcacctttcc

SEQ ID NO: 36 - DNA, 29 bp, Artificial
  HIV-1 Primer
cacaattttaaaagaaaaggggggattgg

SEQ ID NO: 37 - DNA, 27 bp, Artificial
  HIV-1 Primer
tttcgggtttattacagggacagcaga

SEQ ID NO: 38 - DNA, 26 bp, Artificial
  HIV-1 Primer
cttaagacagcagtacaaatggcagt

SEQ ID NO: 39 - DNA, 20 bp, Artificial
  HIV-1 Primer
tctctgctgtccctgtaata

SEQ ID NO: 40 - DNA, 24 bp, Artificial
  HIV-1 Primer
tctctgctgtccctgtaataaacc

SEQ ID NO: 41 - DNA, 28 bp, Artificial
  HIV-1 Probe A
gcgagacagcagtacaaatggcactcgc

SEQ ID NO: 42 - DNA, 26 bp, Artificial
  HIV-1 Probe B
cggaacagcagtacaaatggcatccg

SEQ ID NO: 43 - DNA, 30 bp, Artificial
  HIV-1 Probe C
atctcacagcagtacaaatggcagtgagat

SEQ ID NO: 44 - DNA, 28 bp, Artificial
  HIV-1 Probe D
ctccacagcagtacaaatggcagtggag

SEQ ID NO: 45 - DNA, 26 bp, Artificial
  HIV-1 Probe E
ccacagcagtacaaatggcagtgtgg

SEQ ID NO: 46 - DNA, 28 bp, Artificial
  HIV-1 Probe F
cggaacagcagtacaaatggcagttccg

SEQ ID NO: 47 - DNA, 33 bp, Artificial
  HIV-1 Probe G
accgtacagcagtacaaatggcagtattacggt

SEQ ID NO: 48 - DNA, 37 bp, Artificial
  HIV-1 Probe H
ccgtttacagcagtacaaatggcagtattcaaaacgg

SEQ ID NO: 49 - DNA, 46 bp, Artificial
  HIV-1 Probe I
cacagcagtacaaatggcagtattcatccacaattttaatgctgtg

SEQ ID NO: 50 - DNA, 31 bp, Artificial
  HIV-1 Probe J
cttactcacaattttaaaagaaaagagtaag

SEQ ID NO: 51 - DNA, 32 bp, Artificial
  HIV-1 Probe K
cttactcacaattttaaaagaaaaggagtaag

SEQ ID NO: 52 - DNA, 33 bp, Artificial
  HIV-1 Probe L
ctgactcacaattttaaaagaaaagggagtcag

SEQ ID NO: 53 - DNA, 28 bp, Artificial
  HIV-1 Probe M
gtaccacaattttaaaagaaaagggtac

SEQ ID NO: 54 - DNA, 31 bp, Artificial
  HIV-1 Probe O
acgcctaattttcgggtttattacagggcgt

SEQ ID NO: 55 - DNA, 27 bp, Artificial
  HIV-1 Probe P
catcctttggaaaggaccagcaggatg

SEQ ID NO: 56 - DNA, 33 bp, Artificial
  HIV-1 Probe Q
caccctctggaaaggtgaaggggcagtagggtg

SEQ ID NO: 57 - DNA, 30 bp, Artificial
  HIV-1 Probe R
tcgacaaaggtgaaggggcagtagtgtcga

SEQ ID NO: 58 - DNA, 28 bp, Artificial
  HIV-1 Probe A1
gcgagacagcagtacanatggcactcgc

SEQ ID NO: 59 - DNA, 26 bp, Artificial
  HIV-1 Probe B1
cggaacagcagtacanatggcatccg

SEQ ID NO: 60 - DNA, 26 bp, Artificial
  HIV-1 Probe E1
ccacagcagtacanatggcagtgtgg

SEQ ID NO: 61 - DNA, 37 bp, Artificial
  HIV-1 Probe H1
ccgtttacagcagtacanatggcagtattcaaaacgg

SEQ ID NO: 62 - DNA, 26 bp, Artificial
  HIV-1 Probe Eino
ccacagcagtacanatggcagtgtgg

SEQ ID NO: 63 - DNA, 37 bp, Artificial; identical to SEQ ID NO: 61
  HIV-1 Probe Hino

SEQ ID NO: 64 - DNA, 46 bp, Artificial
  HIV-1 Probe lino
cacagcagtacaaatggcagtattcatncacaattttaatgctgtg

SEQ ID NO: 65 - DNA, 46 bp, Artificial
  HIV-1 Probe lino2
cacagcagtacanatggcagtattcatncacaattttaatgctgtg

SEQ ID NO: 66 - DNA, 28 bp, Artificial
  HIV-1 Molecular Beacon Probe
gcgagacgagttcatgagggcagctcgc

SEQ ID NO: 67 - DNA, 189 bp, Artificial
  HIV-1 Target Sequence
attccctacaatccccaaagtcaaggagtagtagaatctatgaataaagaattaaagaaaattataggacaggtaagagatcaggctgaacatcttaagttgcagctgcaagaaggatcgttgaagctgacgagttcatgagggcaggccgctatgatgaaggggggattggggggtacagtgcagggg

(19) United States
(12) Patent Application Publication
(10) Pub. No.: US 2009/0061413 A1
(43) Pub. Date: Mar. 5, 2009

(54) ISOTHERMAL SCREENING OF HIV-1 RELATED NUCLEIC ACIDS

(76) Inventor: Frank G. Salinas, Wheaton, IL (US)

Correspondence Address: MCANDREWS HELD & MALLOY, LTD, 500 WEST MADISON STREET, SUITE 3400, CHICAGO, IL, 60661, US

(21) Appl. No.: 11/679,690

(22) Filed: Feb. 27, 2007

Related U.S. Application Data
(60) Provisional application No. 60/777,167, filed on Feb. 27, 2006.

(51) Int. Cl. C12Q 1/70

(52) U.S. Cl. 435/5

(57) ABSTRACT

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases.

### RELATED APPLICATIONS

The present application is related to and claims priority from U.S. Provisional Patent Application Ser. No. 60/777,167, filed Feb. 27, 2006, the contents of which are hereby incorporated herein by reference in their entirety. Additionally, all cited references in the present application are hereby incorporated by reference in their entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases. In particular, the presently described technology relates to the methods and materials for the detection of Human Immunodeficiency Virus Type 1 (HIV-1) target sequences in a test sample.

Sub-Saharan Africa has just over 10% of the world's population, but is home to more than 60% of all people living with HIV—25.8 million. In 2005, an estimated 3.2 million people in the region became newly infected, while 2.4 million adults and children died of AIDS. Among young people aged 15-24 years, an estimated 4.6% [4.2-5.5%] of women and 1.7% [1.3-2.2%] of men were living with HIV in 2005.

National HIV infection levels in Asia are low compared with some other continents, notably Africa. However, the populations of many Asian nations are so large that even low national HIV prevalence means large numbers of people are living with HIV. Latest estimates show some 8.3 million [5.4 million-12 million] people (2 million [1.3 million-3 million] adult women) were living with HIV in 2005, including the 1.1 million [600 000-2.5 million] people who became newly infected in the past year. AIDS claimed some 520 000 [330 000-780 000] lives in 2005.

The epidemics in Eastern Europe and Central Asia continue to grow and are affecting ever-larger parts of societies in this region. The number of people living with HIV in this region reached an estimated 1.6 million in 2005—an increase of almost twenty-fold in less than ten years. AIDS claimed almost twice as many lives in 2005, compared with 2003, and killed an estimated 62 000 adults and children. Some 270 000 people were newly infected with HIV in the past year. The overwhelming majority of people living with HIV in this region are young; 75% of the reported infections between 2000 and 2004 were in people younger than 30 years (in Western Europe, the corresponding figure was 33%). The patterns of the epidemics are changing in several countries, with sexually transmitted HIV cases comprising a growing share of new diagnoses. In 2004, 30% or more of all new reported HIV infections in Kazakhstan and Ukraine, and 45% or more in Belarus and the Republic of Moldova, were due to unprotected sex. Increasing numbers of women are being affected, many of them acquiring HIV from male partners who became infected when injecting drugs.

The AIDS epidemic claimed an estimated 24 000 [16 000-40 000] lives in the Caribbean in 2005, making it the leading cause of death among adults aged 15-44 years. A total of 300 000 [200 000-510 000] people are living with HIV in the Caribbean, including the 30 000 [17 000-71 000] people who became infected in 2005. In the Caribbean Community (CARICOM) region 240 000 [150 000-450 000] people are living with HIV, including the 25 000 [12 000-65 000] people who acquired the virus in 2005. More than 20 000 [13 000-36 000] people died of AIDS in the past year in this region.

The number of people living with HIV in Latin America has risen to an estimated 1.8 million. In 2005, approximately 66 000 people died of AIDS, and 200 000 were newly-infected. Among young people 15-24 years of age, an estimated 0.4% of women and 0.6% of men were living with HIV in 2005.

The advance of AIDS in the Middle East and North Africa has continued with latest estimates showing that 67 000 people became infected with HIV in 2005. Approximately 510 000 people are living with HIV in this region. An estimated 58 000 adults and children in 2005 died of AIDS-related conditions.

An estimated 74 000 people in Oceania are living with HIV. Although less than 4000 people are believed to have died of AIDS in 2005, about 8200 are thought to have become newly infected with HIV. Among young people 15-24 years of age, an estimated 1.2% of women and 0.4% of men were living with HIV in 2005.

The number of people living with HIV in North America, Western and Central Europe rose to 1.9 million in 2005, with approximately 65 000 people having acquired HIV in the past year. Wide availability of antiretroviral therapy has helped keep AIDS deaths comparatively low, at about 30 000. The estimated number of people living with HIV in the United States of America (USA) at the end of 2003 exceeded one million for the first time. In Canada, just under 58 000 HIV diagnoses had been reported by the end of 2004.

The main challenge is to intensify prevention efforts and adapt them to the changing patterns of the epidemic. To control the spread of the HIV/AIDS epidemic, especially in third world countries lacking adequate molecular diagnostic resources, there is a need for a field based point-of-care molecular diagnostic system for the amplification and detection of the HIV virus.

### BRIEF SUMMARY OF THE INVENTION

One object of the present invention is to provide a molecular diagnostic system comprising methods and materials for the isothermal screening and detection of nucleic acids. Still another object of the present invention is to provide a molecular diagnostic system comprising methods and reagents for the isothermal screening and detection of nucleic acids associated with but not limited to disease, disease predisposition, disease causative agents, and any combination or derivative thereof. A further object of the present invention is to provide a molecular diagnostic system comprising methods and materials for the isothermal screening and detection of nucleic acids associated with HIV.

One or more of the preceding objects, or one or more other objects which will become plain upon consideration of the present specification, are satisfied by the invention described herein.

One aspect of the invention, which satisfies one or more of the above objects, is a test kit having reagents for the isothermal detection of nucleic acids associated with but not limited to disease, disease predisposition, disease causative agents, and any combination or derivative thereof. Another aspect of the invention is a test kit comprising: a strand transferase component; a polymerase component; and one or more primers and/or probes complementary to one or more nucleic acids associated with but not limited to disease, disease predisposition, disease causative agents, and any combination or derivative thereof. One preferred aspect of the present invention is a test kit comprising: a reverse transcriptase, a strand transferase component; a DNA dependent DNA polymerase component; and one or more primers and/or probes complementary to one or more nucleic acids associated with HIV.

### BRIEF DESCRIPTION OF SEVERAL VIEWS OF THE FIGURES

FIG. 1 is a schematic view of one aspect of the isothermal DNA amplification system of the present invention employing one primer complementary to a target nucleic acid, a strand transferase, and a polymerase.

FIG. 2 is a schematic view of another aspect of the isothermal DNA amplification system of the present invention employing two primers complementary to opposite strands and flanking a target nucleic acid, a strand transferase and a polymerase.

### DETAILED DESCRIPTION OF THE INVENTION

The present invention provides methods and materials for the isothermal screening and detection of nucleic acids associated with but not limited to disease, disease predisposition, disease causative agents, and any combination or derivative thereof. As used herein, and without limitation, nucleic acid generally includes any size DNA, RNA, DNA/RNA hybrid, or analog thereof. The nucleic acid can be single stranded, double stranded, or a combination of single and double stranded. As used herein, and without limitation, disease generally includes an impairment of the normal state of the living animal or plant body or one of its parts that interrupts or modifies the performance of the vital functions, is typically manifested by distinguishing signs and symptoms, and is a response to environmental factors (as malnutrition, industrial hazards, or climate), to specific infective agents (as parasites, bacteria, or viruses), to inherent defects of the organism (as genetic anomalies), or to combinations or derivatives of these factors.

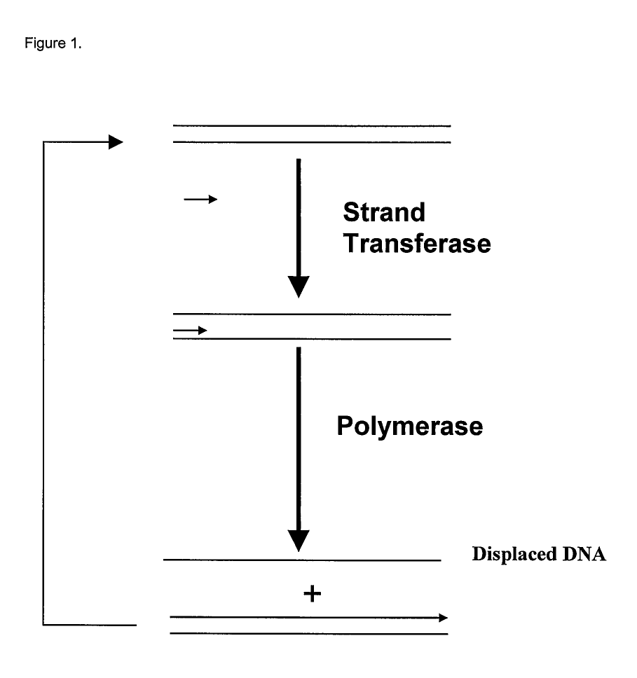

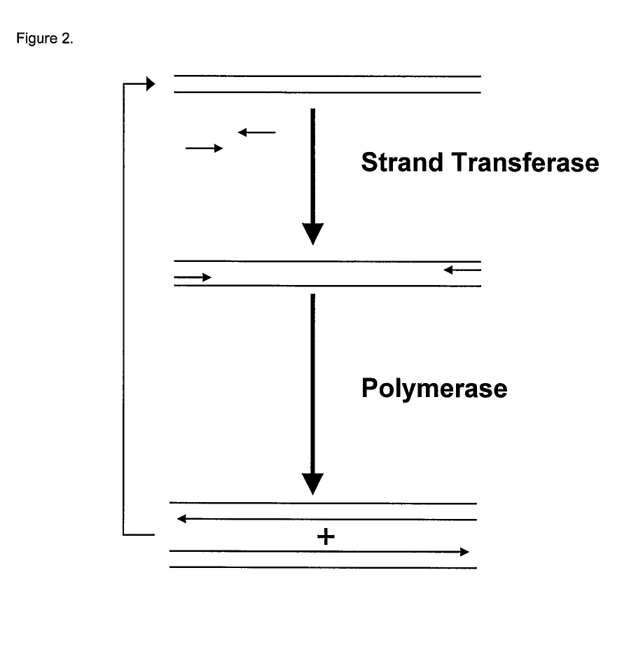

One aspect of the present invention includes methods and materials for the quantitative or qualitative isothermal screening and detection of one or more target nucleic acids of interest. This aspect of the present invention comprises contacting the target nucleic acid with at least one nucleic acid primer having complementarity to the target nucleic acid, a strand transferase, and a polymerase. The strand transferase catalyzes the homologous pairing of the at least one primer to a specific location on the target nucleic acid to form a primer-template junction that is acted upon by the polymerase to replicate and amplify the target nucleic acid (FIG. 1). In one preferred embodiment, the target nucleic acid is contacted with two primers complementary to opposite strands and flanking said target nucleic acid, in the presence of a strand transferase and a polymerase (FIG. 2). In certain aspects of the present invention, the isothermal amplification of the nucleic acid is performed as describe in U.S. Pat. No. 6,929,915, Methods for Nucleic Acid Manipulation. This reference is herein incorporated by reference.

As used herein without limitation, a strand transferase generally is a catalyst for the identification and base pairing of homologous sequences between nucleic acids, a process also known as homologous pairing or strand exchange. Bianco et al provides a general discussion of strand transferases in “DNA strand exchange proteins: a biochemical and physical comparison” at Front Biosci. 1998 Jun. 17; 3:D570-603. This reference is herein incorporated by reference. Strand transferases can be derived from either a prokaryotic system or an eukaryotic system, including but not limited to yeast, bacteria, and bacteriophages such as T4 and T7. For example West discusses eukaryotic strand transferases in Recombination genes and proteins” in Curr Opin Genet Dev. 1994 April; 4(2):221-8. This reference is herein incorporated by reference. Radding discussed the recA strand exchange protein in “Helical RecA nucleoprotein filaments mediate homologous pairing and strand exchange” at Biochim Biophys Acta. 1989 Jul. 7; 1008(2):131-45. This reference is herein incorporated by reference. Also, the UvsX strand transferase was described by Kodadek et al., The mechanism of homologous DNA strand exchange catalyzed by the bacteriophage T4 uvsX and gene 32 proteins” JBC 1988 Jul. 5; 263(19):9427-36. This reference is herein incorporated by reference. Yonesaki discusses T4 homologous recombination in “Recombination apparatus of T4 phage” at Adv Biophys. 1995; 31:3-22. This reference is herein incorporated by reference. Also, Salinas et. al have discussed the homology dependence of UvsX catalyzed strand exchange in “Homology dependence of UvsX protein-catalyzed joint molecule formation” at J Biol. Chem. 1995 Mar. 10; 270(10):5181-6. This reference is herein incorporated by reference. Exemplar strand transferase proteins include but are not limited to the eukaryotic Rad51 protein, the bacterial recA protein, the bacterial phage T4 UvsX protein, the bacteriophage T7 gene 2.5 or any protein fragment, derivative, or homolog thereof, including proteins found in nature and those engineered or modified using recombinant DNA technology. Kong et. al has discussed T7 strand exchange in “Role of the bacteriophage T7 and T4 single-stranded DNA-binding proteins in the formation of joint molecules and DNA helicase-catalyzed polar branch migration.” J Biol. Chem. 1997 Mar. 28; 272(13):8380-7. This reference is herein incorporated by reference.

Strand transferases generally operate by first binding single stranded regions of DNA to form a nucleoprotein filament generally referred to as the presynaptic filament. The presynaptic filament then binds a target nucleic acid and performs a search for homology that once complete results in the formation of a joint molecule or D-loop. Strand transferases generally have accessory protein factors that augment or modify their activity. For example, strand transferases generally have accessory protein factors that effect the formation and/or stability of the presynaptic filament under varying conditions, including for example buffer conditions and/or the presence of other proteins competing to bind regions of single-stranded nucleic acid. Exemplar strand transferase accessory proteins include but are not limited to the bacteriophage T4 UvsX accessory protein UvsY, the E. coli RecA accessory proteins RecFOR, the yeast and human Rad51 accessory protein Rad52, and any protein fragment, derivative, or homolog thereof, including proteins found in nature and those engineered or modified using recombinant DNA technology.

As used herein without limitation, a polymerase generally is any of several enzymes, such as DNA polymerase, RNA polymerase, or reverse transcriptase, that catalyze the formation of nucleic acid from precursor substances in the presence of preexisting nucleic acid acting as a template. The polymerase of the present invention can be derived from a eukaryotic or a prokaryotic system. For example the polymerase can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar polymerases include but are not limited to the bacteriophage T4 gene product 43 protein, and any mutants or derivatives of the gene 43 protein including but not limited to the exonuclease deficient 43 exo⁻ polymerase. Benkovic et. al discusses replisome mediated DNA replication in “Replisome Mediated DNA Replication” at Annu Rev Biochem. 2001; 70:181-208. This reference is herein incorporated by reference.

Polymerases generally have accessory protein factors that augment or modify their activity. Exemplar polymerase accessory factors include but are not limited to clamp proteins and clamp loader proteins. Clamp proteins generally have affinity and/or a topological link to both the polymerase and the nucleic acid being acted upon by said polymerase, thereby forming a stable link between polymerase and nucleic acid, the result of which is the formation of a stable polymerase nucleic acid complex having high processivity Clamp loader proteins facilitate the assembly of a clamp protein onto a nucleic acid and can also facilitate and mediate a concomitant or subsequent interaction with the polymerase. As used herein in connection with certain aspects and embodiments of the invention, the term holoenzyme generally regards a polymerase-clamp complex.

Polymerase accessory factors can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar clamp proteins include but are not limited to the bacteriophage T4 gene product 45 protein, and any mutants or derivatives of the T4 gene product 45 protein. Trakselis et discuss the T4 polymerase holoenzyme in Creating a dynamic picture of the sliding clamp during T4 DNA polymerase holoenzyme assembly by using fluorescence resonance energy transfer” at Proc Natl Acad Sci USA. 2001 Jul. 17; 98(15):8368-75. This reference is herein incorporated by reference.

In certain embodiments of the present invention, the quantitative or qualitative isothermal screening and detection of one or more target nucleic acids of interest is performed in the presence of a single stranded nucleic acid binding protein (SSB). SSB's used pursuant to the present invention can be derived from a bacterium such as E. coli, a bacteriophage such as bacteriophage T4 or bacteriophage T7, a eukaryotic organism such as yeast or human, or any protein fragment, derivative, or homolog thereof, including proteins found in nature and those engineered or modified using recombinant DNA technology. Exemplar SSB's include but are not limited to the E. coli SSB protein, the bacteriophage T4 gene product 32 protein, the bacteriophage T7 gene product 2.5 protein, and the yeast or human RPA protein, or any mutants or derivatives thereof.

In certain embodiments of the present invention, the quantitative or qualitative isothermal screening and detection of one or more target nucleic acids of interest is performed in the presence of a helicase, preferably a DNA helicase. The helicase can be derived from a prokaryote or a eukaryote. For example, the DNA helicase can be from a bacterium such as E. coli., a bacteriophage such as bacteriophage T4 or bacteriophage T7, a yeast, or human. Exemplar helicases include but are not limited to the bacteriophage T4 gene product 41, the bacteriophage T4 dda protein, the bacteriophage T7 gene 4 protein, the E. coli UvrD protein, and any mutants or derivatives thereof. For example, Salinas and Kodadek have discussed the role of DNA helicases during strand homologous recombination in “Phage T4 homologous strand exchange: a DNA helicase, not the strand transferase, drives polar branch migration.” Cell 1995 Jul. 14; 82(1):111-9. This reference is herein incorporated by reference. Also, Salinas and Benkovic have discussed the role of DNA helicases in bacteriophage T4 replication in “Characterization of bacteriophage T4-coordinated leading- and lagging-strand synthesis on a minicircle substrate.” Proc Natl Acad Sci USA. 2000 Jun. 20; 97(13):7196-201. This reference is herein incorporated by reference. Also, Alberts et al discusses the general nature of replication in bacteriophage T4 in “Studies on DNA replication in the bacteriophage T4 in vitro system” at Cold Spring Harb Symp Quant Biol. 1983; 47 Pt 2:655-68. This reference is herein incorporated by reference.

In certain other embodiments of the present invention, the quantitative or qualitative isothermal screening and detection of one or more target nucleic acids of interest is performed in the presence of a helicase and a helicase accessory factor. The DNA helicase and the DNA helicase accessory factor can be derived from a eukaryotic or prokaryotic system. For example, the DNA helicase and the DNA helicase accessory factor can be from a bacterial system such as E. coli. or a bacteriophage system such as bacteriophage T4. For example, one DNA helicase/accessory factor pair is the bacteriophage T4 gene product 41 protein and its accessory factor gene product 59 protein. Jones et al discusses the gene product 59 protein in “Bacteriophage T4 gene 41 helicase and gene 59 helicase-loading protein: a versatile couple with roles in replication and recombination” at Proc Natl Acad Sci USA. 2001 Jul. 17; 98(15):8312-8. This reference is herein incorporated by reference.

In still other embodiments of the present invention, the quantitative or qualitative isothermal screening and detection of one or more target nucleic acids of interest is performed in the presence of a primosome. As used herein a primosome is a term that generally characterizes a complex comprising a DNA helicase and an RNA polymerase usually referred to as a primase. The primosome is active in synthesizing RNA primers on the lagging strand of a replication fork for the initiation of Okazaki fragment synthesis during coordinated leading- and lagging strand synthesis. Primases can be derived from a prokaryote or a eukaryote. For example, the primase can be from a bacterium such as E. coli., a bacteriophage such as bacteriophage T4 or bacteriophage T7, a yeast, or a human. One exemplar primase is the bacteriophage T4 gene product 61 protein, and derivatives or mutants thereof.

The phrase “amplification reaction reagents” as used herein includes but is not limited to reagents which are well known for their use in nucleic acid amplification reactions and may include but are not limited to: a single or multiple reagent, reagents, enzyme or enzymes separately or individually having reverse transcriptase and/or polymerase activity, strand transferase activity, or exonuclease activity; enzyme cofactors such as magnesium or manganese; salts; nicotinamide adenine dinucleotide (NAD); and deoxynucleoside triphosphates (dNTPs) such as, for example, deoxyadenosine triphosphate, deoxyguanosine triphosphate, deoxycytodine triphosphate and thymidine triphosphate. Other reagents include molecular crowding agents, including but not limited to polyethylene glycol PEG 8000. The exact amplification reagents employed are largely a matter of choice for one skilled in the art based upon the particular amplification reaction employed. For example, it is known in the art that volume occupying agents, or molecular crowding agents, enhance the activity or function of strand transferases, polymerases, and their accessory factors. The following references are herein incorporated by reference: (1) “Enhancement of recA Protein-promoted DNA Strand Exchange Activity by Volume occupying agents” at J Biol. Chem. 1992 May 5; 267(13):9307-14; (2) “Stimulation of the processivity of the DNA polymerase of bacteriophage T4 by the polymerase accessory proteins” at J Biol. Chem. 1991 Jan. 25; 266(3):1830-40; (3) “Macromolecular crowding”: thermodynamic consequences for protein-protein interactions within the T4 DNA replication complex: The role of ATP hydrolysis”; (4) “Macromolecular crowding”: thermodynamic consequences for protein-protein interactions within the T4 DNA replication complex” at J Biol. Chem. 1990 Sep. 5; 265(25):15160-7; (5) “Assembly of a functional replication complex without ATP hydrolysis: a direct interaction of bacteriophage T4 gp45 with T4 DNA polymerase” at Proc Natl Acad Sci USA. 1993 Apr. 15; 90(8):3211-5; and (6) “A coupled complex of T4 DNA replication helicase (gp41) and polymerase (gp43) can perform rapid and processive DNA strand-displacement synthesis” at Proc Natl Acad Sci USA. 1996 Dec. 10; 93(25):14456-61.

### Target Nucleic Acids

Target nucleic acids of the present invention include but are not limited to those nucleic acids associated with the development or onset of a disease state, including for example those nucleic acids that show the presence of specific infective agents or inherent defects of in an organism's genome. Target nucleic acids include but are not limited to either or both nucleic acids exogenous or endogenous to the organism being screened. Exemplar target nucleic acids belonging to specific infective agents of interest include but are not limited to those nucleic acids derived from protozoa, parasites, fingi, bacteria, viruses, and combinations or derivatives thereof.

An object of the present invention is to provide methods and materials for the isothermal screening and detection of nucleic acids associated with HIV-1. The present invention provides reagents useful for the isothermal amplification and detection of all HIV-1 group M, N, and O strains including CRF and inter-group recombinants. Primer sets, probes, and reagents for specifically and sensitively amplifying and detecting HIV-1 variants including those from HIV-1 groups M, N, and O, as well as the various subtypes within or derived from these groups has been described in U.S. Pat. No. 6,852,491, Amplification and detection reagents for HIV-1. This reference is herein incorporated by reference.

Primer sets of the present invention that can be utilized to detect HIV-1 are designated herein as primer set 1 (SEQ. ID. NO. 1 and SEQ. ID. NO. 2); primer set 2 (SEQ. ID. NO. 3 and SEQ. ID. NO. 2); primer set 3 (SEQ. ID. NO. 4 and SEQ. ID. NO. 2); primer set 4 (SEQ. ID. NO. 5 and SEQ. ID. NO. 2); primer set 5 (SEQ. ID. NO. 6 and SEQ. ID. NO. 2); primer set 6 (SEQ. ID. NO. 7 and SEQ. ID. NO. 2); primer set 7 (SEQ. ID. NO. 8 and SEQ. ID. NO. 2); primer set 8 (SEQ. ID. NO. 9 and SEQ. ID. NO. 10); primer set 9 (SEQ. ID. NO. 9 and SEQ. ID. NO. 11); primer set 10 (SEQ. ID. NO. 1 and SEQ. ID. NO. 12); primer set 11 (SEQ. ID. NO. 13 and SEQ. ID. NO. 14); primer set 12 (SEQ. ID. NO. 13 and SEQ. ID. NO. 15); primer set 13 (SEQ. ID. NO. 13 and SEQ. ID. NO. 2); primer set 14 (SEQ. ID. NO. 9 and SEQ. ID. NO. 12); primer set 15 (SEQ. ID. NO. 1 and SEQ. ID. NO. 11); primer set 16 (SEQ. ID. NO. 16 and SEQ. ID. NO. 12); primer set 17 (SEQ. ID. NO. 16 and SEQ. ID. NO. 17); primer set 18 (SEQ. ID. NO. 3 and SEQ. ID. NO. 12); primer set 19 (SEQ. ID. NO. 3 and SEQ. ID. NO. 18); primer set 20 (SEQ. ID. NO. 19 and SEQ. ID. NO. 18); primer set 21 (SEQ. ID. NO. 13 and SEQ. ID. NO. 17); primer set 22 (SEQ. ID. NO. 13 and SEQ. ID. NO. 20); primer set 23 (SEQ. ID. NO. 21 and SEQ. ID. NO. 18); primer set 24 (SEQ. ID. NO. 21 and SEQ. ID. NO. 14); primer set 25 (SEQ. ID. NO. 21 and SEQ. ID. NO. 20); primer set 26 (SEQ. ID. NO. 4 and SEQ. ID. NO. 20); primer set 27 (SEQ. ID. NO. 5 and SEQ. ID. NO. 15); primer set 28 (SEQ. ID. NO. 21 and SEQ. ID. NO. 22); primer set 29 (SEQ. ID. NO. 21 and SEQ. ID. NO. 23); primer set 30 (SEQ. ID. NO. 5 and SEQ. ID. NO. 23); primer set 31 (SEQ. ID. NO. 28 and SEQ. ID. NO. 29); primer set 32 (SEQ. ID. NO. 28 and SEQ. ID. NO. 30); primer set 33 (SEQ. ID. NO. 28 and SEQ. ID. NO. 31); primer set 38 (SEQ. ID. NO. 37 and SEQ. ID. NO. 32); primer set 39 (SEQ. ID. NO. 37 and SEQ. ID. NO. 33); primer set 40 (SEQ. ID. NO. 38 and SEQ. ID. NO. 29); primer set 41 (SEQ. ID. NO. 38 and SEQ. ID. NO. 30); primer set 42 (SEQ. ID. NO. 4 and SEQ. ID. NO. 22); primer set 43 (SEQ. ID. NO. 4 and SEQ. ID. NO. 40); primer set 44 (SEQ. ID. NO. 34 and SEQ. ID. NO. 22); primer set 45 (SEQ. ID. NO. 34 and SEQ. ID. NO. 40); primer set 46 (SEQ. ID. NO. 24 and SEQ. ID. NO. 25); and primer set 47 (SEQ. ID. NO. 26 and SEQ. ID. NO. 27).

The probe sequences provided that may be employed to detect an HIV-1 target sequence (whether amplified or not) are designated herein as SEQ. ID. NO. 41; SEQ. ID. NO. 42; SEQ. ID. NO. 43; SEQ. ID. NO. 44; SEQ. ID. NO. 45; SEQ. ID. NO. 47; SEQ. ID. NO. 48; SEQ. ID. NO. 49; SEQ. ID. NO. 50; SEQ. ID. NO. 51; SEQ. ID. NO. 52; SEQ. ID. NO. 53; SEQ. ID. NO. 55; SEQ. ID. NO. 57; SEQ. ID. NO. 58; SEQ. ID. NO. 59; SEQ. ID. NO. 60; SEQ. ID. NO. 61; SEQ. ID. NO. 62; SEQ. ID. NO. 63; SEQ. ID. NO. 64; and SEQ. ID. NO. 65.

Oligo sets that can be used to amplify and detect an HIV-1 target sequence are designated herein as oligo set 1 (SEQ. ID. NO. 28, SEQ. ID. NO. 29, and SEQ. ID. NO. 41); oligo set 2 (SEQ. ID. NO. 28, SEQ. ID. NO. 29, and SEQ. ID. NO. 42); oligo set 3 (SEQ. ID. NO. 28, SEQ. ID. NO. 29, and SEQ. ID. NO. 43); oligo set 4 (SEQ. ID. NO. 28, SEQ. ID. NO. 29, and SEQ. ID. NO. 44); oligo set 5 (SEQ. ID. NO. 28, SEQ. ID. NO. 29, and SEQ. ID. NO. 45); oligo set 7 (SEQ. ID. NO. 28, SEQ. ID. NO. 29, and SEQ. ID. NO. 47); oligo set 8 (SEQ. ID. NO. 28, SEQ. ID. NO. 29, and SEQ. ID. NO. 48); oligo set 9 (SEQ. ID. NO. 28, SEQ. ID. NO. 29, and SEQ. ID. NO. 49); oligo set 10 (SEQ. ID. NO. 28, SEQ. ID. NO. 29, and SEQ. ID. NO. 50); oligo set 11 (SEQ. ID. NO. 28, SEQ. ID. NO. 29, and SEQ. ID. NO. 51); oligo set 12 (SEQ. ID. NO. 28, SEQ. ID. NO. 29, and SEQ. ID. NO. 52); oligo set 13 (SEQ. ID. NO. 28, SEQ. ID. NO. 29, and SEQ. ID. NO. 53); oligo set 14 (SEQ. ID. NO. 28, SEQ. ID. NO. 30, and SEQ. ID. NO. 41); oligo set 15 (SEQ. ID. NO. 28, SEQ. ID. NO. 30, and SEQ. ID. NO. 42); oligo set 16 (SEQ. ID. NO. 28, SEQ. ID. NO. 30, and SEQ. ID. NO. 43); oligo set 17 (SEQ. ID. NO. 28, SEQ. ID. NO. 30, and SEQ. ID. NO. 44); oligo set 18 (SEQ. ID. NO. 28, SEQ. ID. NO. 30, and SEQ. ID. NO. 45); oligo set 20 (SEQ. ID. NO. 28, SEQ. ID. NO. 30, and SEQ. ID. NO. 47); oligo set 21 (SEQ. ID. NO. 28, SEQ. ID. NO. 30, and SEQ. ID. NO. 48); oligo set 22 (SEQ. ID. NO. 28, SEQ. ID. NO. 30, and SEQ. ID. NO. 49); oligo set 23 (SEQ. ID. NO. 28, SEQ. ID. NO. 30, and SEQ. ID. NO. 50); oligo set 24 (SEQ. ID. NO. 28, SEQ. ID. NO. 30, and SEQ. ID. NO. 51); oligo set 25 (SEQ. ID. NO. 28, SEQ. ID. NO. 30, and SEQ. ID. NO. 52); oligo set 26 (SEQ. ID. NO. 28, SEQ. ID. NO. 30, and SEQ. ID. NO. 53); oligo set 27 (SEQ. ID. NO. 28, SEQ. ID. NO. 31, and SEQ. ID. NO. 41); oligo set 28 (SEQ. ID. NO. 28, SEQ. ID. NO. 31, and SEQ. ID. NO. 42); oligo set 29 (SEQ. ID. NO. 28, SEQ. ID. NO. 31, and SEQ. ID. NO. 43); oligo set 30 (SEQ. ID. NO. 28, SEQ. ID. NO. 31, and SEQ. ID. NO. 44); oligo set 31 (SEQ. ID. NO. 28, SEQ. ID. NO. 31, and SEQ. ID. NO. 45); oligo set 33 (SEQ. ID. NO. 28, SEQ. ID. NO. 31, and SEQ. ID. NO. 47); oligo set 34 (SEQ. ID. NO. 28, SEQ. ID. NO. 31, and SEQ. ID. NO. 48); oligo set 35 (SEQ. ID. NO. 37, SEQ. ID. NO. 32, and SEQ. ID. NO. 55); oligo set 36 (SEQ. ID. NO. 37, SEQ. ID. NO. 33, and SEQ. ID. NO. 55); oligo set 38 (SEQ. ID. NO. 37, SEQ. ID. NO. 33, and SEQ. ID. NO. 57); oligo set 39 (SEQ. ID. NO. 38, SEQ. ID. NO. 29, and SEQ. ID. NO. 50); oligo set 40 (SEQ. ID. NO. 38, SEQ. ID. NO. 29, and SEQ. ID. NO. 51); oligo set 41 (SEQ. ID. NO. 38, SEQ. ID. NO. 29, and SEQ. ID. NO. 52); oligo set 42 (SEQ. ID. NO. 38, SEQ. ID. NO. 29, and SEQ. ID. NO. 53); oligo set 43 (SEQ. ID. NO. 38, SEQ. ID. NO. 30, and SEQ. ID. NO. 50); oligo set 44 (SEQ. ID. NO. 38, SEQ. ID. NO. 30, and SEQ. ID. NO. 51); oligo set 45 (SEQ. ID. NO. 38, SEQ. ID. NO. 30, and SEQ. ID. NO. 52); oligo set 46 (SEQ. ID. NO. 38, SEQ. ID. NO. 30, and SEQ. ID. NO. 53); oligo set 47 (SEQ. ID. NO. 28, SEQ. ID. NO. 30, and SEQ. ID. NO. 58); oligo set 48 (SEQ. ID. NO. 28, SEQ. ID. NO. 30, and SEQ. ID. NO. 59); oligo set 49 (SEQ. ID. NO. 28, SEQ. ID. NO. 30, and SEQ. ID. NO. 60); oligo set 50 (SEQ. ID. NO. 28, SEQ. ID. NO. 30, and SEQ. ID. NO. 61); oligo set 51 (SEQ. ID. NO. 28, SEQ. ID. NO. 30, and SEQ. ID. NO. 62); oligo set 52 (SEQ. ID. NO. 28, SEQ. ID. NO. 30, and SEQ. ID. NO. 63); oligo set 53 (SEQ. ID. NO. 28, SEQ. ID. NO. 30, and SEQ. ID. NO. 64); and oligo set 54 (SEQ. ID. NO. 28, SEQ. ID. NO. 30, and SEQ. ID. NO. 65).

The primer sets provided herein can be employed according to the isothermal DNA amplification disclosed herein. Probe sequences are also provided. The probe sequences can be combined with various primer sets to form oligonucleotide or “oligo” sets that can be used to amplify and detect an HIV-1 target sequence.

Methods for amplifying and detecting HIV-1 in a test sample generally comprise contacting a test sample with a strand transferase, a polymerase, amplification reagents and a previously mentioned primer set to form a reaction mixture. The reaction mixture is then placed under amplification conditions to form an amplification product to thereby amplify the HIV-1 target sequence. Amplification products may be detected using a variety of detection technologies. Preferably, however, an amplification product/probe hybrid is formed and detected as an indication of the presence of HIV-1 in the test sample.

The primer sets provided herein comprise two oligonucleotide primers that can be employed to amplify an HIV-1 target sequence in a test sample. The term “test sample” as used herein, means anything suspected of containing an HIV-1 target sequence. The test sample is, or can be derived from, any biological source, such as for example, blood, seminal fluid, ocular lens fluid, cerebral spinal fluid, milk, ascites fluid, synovial fluid, peritoneal fluid, amniotic fluid, tissue, fermentation broths, cell cultures and the like. The test sample can be used (i) directly as obtained from the source or (ii) following a pre-treatment to modify the character of the sample. Thus, the test sample can be pre-treated prior to use by, for example, preparing plasma from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

A “target sequence” as used herein includes but is not limited to a nucleic acid sequence that is amplified, detected, or both amplified and detected using the primer sets herein provided. Additionally, while the term target sequence is sometimes referred to as single stranded, those skilled in the art will recognize that the target sequence may actually be double stranded. Thus, in cases where the target is double stranded, primer sequences of the present invention will amplify both strands of the target sequence.

The primer sets that can be employed to amplify an HIV-1 target sequence preferably comprise deoxyribonucleic acid (DNA), or ribonucleic acid (RNA). Such primer sets can be employed according to isothermal DNA amplification disclosed herein. Additionally, in light of the RNA nature of the HIV-1 genome, the isothermal nucleic acid amplification technology disclosed herein, and the primer sets may be employed in combination with a reverse transcriptase. Briefly, the reverse transcriptase provides a method of transcribing a strand of DNA from an RNA target sequence. The copied DNA strand transcribed from the RNA target is commonly referred to as “cDNA” which then can serve as a template for amplification by the isothermal nucleic acid amplification system mentioned above. The process of generating cDNA shares many of the hybridization and extension principles surrounding the isothermal nucleic acid amplification system described herein, but at least one of the enzymes employed should have reverse transcriptase activity. Enzymes having reverse transcriptase activity are well known and therefore don't warrant further discussion. Additionally, other methods for synthesizing cDNA are also known and include commonly owned U.S. patent application Ser. No. 08/356,287 filed Feb. 22, 1995, which is herein incorporated by reference. Generally, therefore, amplifying an HIV-1 target sequence in a test sample will generally comprise the steps of contacting a test sample with a primer set, a strand transferase, a polymerase, and amplification reagents to form a reaction mixture and placing the reaction mixture under amplification conditions to thereby amplify the target sequence.

Amplification products produced using the primer sets provided herein may be detected using a variety of detection technologies well known in the art. For example, amplification products may be detected using agarose gel electrophoresis and visualization by ethidium bromide staining and exposure to Ultraviolet (UV) light or by sequence analysis of the amplification product for confirmation of HIV-1 identity.

Alternatively, amplification products may be detected by oligonucleotide hybridization with a probe. Probe sequences generally are 10 to 50 nucleotides long, more typically 15 to 40 nucleotides long, and similarly to primer sequences, probe sequences are also nucleic acid. Hence, probes may comprise DNA, RNA or nucleic acid analogs such as uncharged nucleic acid analogs including but not limited to peptide nucleic acids (PNAs) which are disclosed in International Patent Application WO 92/20702 or morpholino analogs which are described in U.S. Pat. Nos. 5,185,444, 5,034,506, and 5,142,047 all of which are herein incorporated by reference. Such sequences can routinely be synthesized using a variety of techniques currently available. For example, a sequence of DNA can be synthesized using conventional nucleotide phosphoramidite chemistry and the instruments available from Applied Biosystems, Inc, (Foster City, Calif.); DuPont, (Wilmington, Del.); or Milligen, (Bedford, Mass.). Similarly, and when desirable, all nucleic acids disclosed herein, including but not limited to amplified target nucleic acids, primers, probes, or any combination thereof can be labeled using methodologies well known in the art such as described in U.S. Pat. Nos. 5,464,746; 5,424,414; and 4,948,882 all of which are herein incorporated by reference. Additionally, probes typically hybridize with the target sequence between the primer sequences. In other words, the probe sequence typically is not coextensive with either primer.

The term “label” as used herein means a molecule or moiety having a property or characteristic which is capable of detection. A label can be directly detectable, as with, for example, radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, light, and the like to enable detection of the label. When indirectly detectable labels are used, they are typically used in combination with a “conjugate”. A conjugate is typically a specific binding member which has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

Probe sequences can be employed using a variety of homogeneous or heterogeneous methodologies to detect amplification products. Generally all such methods employ a step where the probe hybridizes to a strand of an amplification product to form an amplification product/probe hybrid. The hybrid can then be detected using labels on the amplified product, the primer, the probe or any combination thereof. Examples of homogeneous detection platforms for detecting amplification products include the use of FRET (fluorescence resonance energy transfer) labels attached to probes that emit a signal in the presence of the target sequence. So-called TaqMan assays described in U.S. Pat. No. 5,210,015 (herein incorporated by reference) and Molecular Beacon assays described in U.S. Pat. No. 5,925,517 (herein incorporated by reference) are examples of techniques that can be employed to homogeneously detect nucleic acid sequences. According to homogenous detection techniques, products of the amplification reaction can be detected as they are formed or in a so-called real time manner. As a result, amplification product/probe hybrids are formed and detected while the reaction mixture is under amplification conditions.

Heterogeneous detection formats typically employ a capture reagent to separate amplified sequences from other materials employed in the reaction. Capture reagents typically are a solid support material that is coated with one or more specific binding members specific for the same or different binding members. A “solid support material”, as used herein, refers to any material which is insoluble, or can be made insoluble by a subsequent reaction. Solid support materials thus can be a latex, plastic, derivatized plastic, magnetic or non-magnetic metal, glass or silicon surface or surfaces of test tubes, microtiter wells, sheets, beads, microparticles, chips, and other configurations known to those of ordinary skill in the art. To facilitate detection of an amplification product/probe hybrid in a heterogeneous type manner, primer or probes or both can be labeled with a first binding member which is specific for its binding partner which is attached to a solid support material such as a microparticle. Similarly, primers may be labeled with a second binding member specific for a conjugate as defined above. The amplification products bound to the probes can then be separated from the remaining reaction mixture by contacting the reaction mixture with the above solid support and then removing the solid support from the reaction mixture. Any amplification product/probe hybrids bound to the solid support may then be contacted with a conjugate to detect the presence of the hybrids on the solid support.

Whether detected in a homogeneous or heterogeneous manner, methods for detecting a target sequence in a test sample will generally comprise the steps of contacting a test sample with a primer set provided herein, a strand transferase, a polymerase, and amplification reagents to form a reaction mixture. The reaction mixture then is placed under amplification conditions to form an amplification product, as specified above. The amplification product is then detected as an indication of the presence of the target sequence in the test sample. As stated above, the reaction product may be detected using gel electrophoresis, heterogeneous methods or homogeneous methods. Accordingly, the reaction product may be detected in the reaction mixture while it is under amplification conditions with homogeneous techniques. Alternatively, the amplification product may be detected after amplification of the target sequence is complete using heterogeneous techniques or gels.

The present invention also provides oligonucleotide sets useful for amplifying and detecting an HIV-1 target sequence in a test sample. These oligonucleotide sets, or “oligo sets”, comprise a primer set and a molecular beacon probe that can be used in the manner set forth above. Additionally, the oligo sets may be packaged in suitable containers and provided with additional reagents such as, for example, amplification reagents (also in suitable containers) to provide kits for detecting HIV-1 in a test sample.

In one embodiment of the present invention, a test sample is reacted with a primer set either alone or in combination with other primer sets in a multiplex reaction. The test sample and the primers are reacted in combination with a strand transferase, a polymerase, and amplification reagents to produce an amplified target nucleic acid that can be detected either by detection of a labeled probe, or by detection of a labeled incorporated nucleotide, or by detection of a labeled primer, or by a combination thereof.

In another embodiment of the present invention, a test sample is reacted with a single primer, or with two or more single primers selected from different primer sets in a multiplex reaction, whereby said primer or primers are complementary to the same strand of nucleic acid. The test sample and the primers can be reacted in combination with a strand transferase, a polymerase, and amplification reagents to produce amplified single stranded target nucleic acid that can be detected either by the detection of a labeled probe, or by the detection of an labeled incorporated nucleotide, or by the detection of a labeled primer, or by a combination thereof.

In certain embodiments of the present invention, a target nucleic acid is detected by reacting a test sample and any one or more primers or primer sets with a reverse transcriptase polymerase to produce a first cDNA. Reaction of the test sample and the primer or primers with the reverse transcriptase to produce a cDNA is performed either before or concomitant with the admixture of a strand transferase, a polymerase, and amplification reagents. The amplified target nucleic acid can be detected either by the detection of a labeled probe, or by the detection of an labeled incorporated nucleotide, or by the detection of a labeled primer, or by a combination thereof.

While the invention has been described in detail and with reference to specific embodiments, it will be apparent to one skilled in the art that various changes and modifications may be made to such embodiments without departing from the spirit and scope of the invention.

What is claimed is:

1. A method for detecting the presence of HIV-1 in a test sample comprising contacting a test sample with a reverse transcriptase, a strand transferase, a DNA dependent DNA polymerase, and at least one primer having complementarity to HIV-1 nucleic acid.

2. The method of claim 1 wherein said strand transferase is derived from a prokaryotic.

3. The method of claim 1 wherein said strand transferase is the uvsX strand transferase derived from the bacteriophage T4.

4. The polymerase of claim 1 wherein said DNA dependent DNA polymerase is derived from a prokaryotic.

5. The polymerase of claim 1 wherein said DNA dependent DNA polymerase is the gp43 polymerase derived from the bacteriophage T4.
